(19) United States
(12) Patent Application Publication
(10) Pub. No.: US 2009/0198276 A1
(43) Pub. Date: Aug. 6, 2009

(54) PELVIC BALANCER

(75) Inventor: Young-Jun Lee, Cheonan-si (KR)

Correspondence Address: AMPACC LAW GROUP, 3500 188th St. SW, Lynnwood, WA, 98037, US

(73) Assignee: JinBiotech Co., Ltd, Cheonan-si (KR)

(21) Appl. No.: 12/364,424

(22) Filed: Feb. 2, 2009

(30) Foreign Application Priority Data
Feb. 2, 2008 (KR) 20-2008-0001603
Jan. 23, 2009 (KR) 20-2009-0000872

(51) Int. Cl. A61F 5/00

(52) U.S. Cl. 606/237

(57) ABSTRACT

A pelvic balancer is disclosed. The pelvic balancer includes a first correction part to support any one of user's left and right pelvic bones placed thereon, a second correction part to support the other one of the user's left and right pelvic bones placed thereon, and a central shaft connected with the first correction part and the second correction part so that the first correction part and the second correction part can rotate. Accordingly, pelvis correction can be achieved by setting a dislocated pelvis to a normal position only by using a user's weight without application of a physical pressing force.

### BACKGROUND OF THE INVENTION

The present invention relates to a pelvic balancer, and more particularly, to a pelvic balancer which achieves pelvic correction by setting a misaligned pelvis to a normal position by using a user's own weight.

A spinal column, serving as a supporting axis for a human body, may be twisted due to a crooked posture, lack of exercise, excessive stress, etc. The twist of a spinal column may result in misalignment of the pelvis.

The misaligned pelvis may affect muscles or nervous systems connected to the pelvis, which causes various symptoms. This misalignment may cause back pain by pressing against the spinal column, and may cause indigestion or stomachache by hindering internal organs from performing their essential functions. Also, the misaligned pelvis may cause the length of each leg to be different, which hinders nervous systems and circulatory systems from performing their functions, resulting in pelvis pain, pulled leg muscles, arthritis, neuralgia, etc.

Techniques to correct the misalignment of a pelvis are disclosed in U.S. Pat. Nos. 7,278,979, 6,352,074 and 5,913,410. The conventional techniques commonly use a pelvis correction method of forcibly setting a misaligned pelvis to a normal position through application of a physical pressing force using a mechanical machine, a belt, etc. However, such conventional techniques have shortcomings such as a long time is necessary for pelvis correction and a patient may feel severe pain.

Besides the above-described conventional techniques, the help of a physical therapist's can be used, where the therapist applies a physical pressing force to a patient. However, such a pelvis correction method has shortcomings in that there is proficiency difference between physical therapists and a patient may feel severe pain. Further, symptoms may become worse due to a physical therapist's error of judgment or poor medical treatment.

### SUMMARY OF THE INVENTION

Therefore, in view of the above issues, the present invention relates to a pelvic balancer which sets a misaligned pelvis to a normal position by using a user's weight and a lever system.

The present invention also relates to a pelvic balancer which includes rotatable first and second correction parts, on which a left pelvic bone and a right pelvic bone are placed so as to apply pressure by a user's own weight, to thereby correct imbalance of the left pelvic bone and the right pelvic bone.

The present invention further relates to a pelvic balancer which can maximize pelvic correction effect by setting left and right pelvic bones to normal positions only by simple manipulation without a sophisticated technique, with a first correction part and a second correction part having the same rotational radius and a center line formed on a central shaft provided at a joining portion of the first correction part and the second correction part.

Additional objects and/or advantages of the invention will be set forth in part in the description which follows and, in part, will be obvious from the description, or may be learned by practice of the invention.

To achieve these objects and other advantages and in accordance with the purpose of the invention, as embodied and broadly described herein, a pelvic balancer includes: a first correction part to support any one of user's left and right pelvic bones placed thereon; a second correction part to support the other one of the user's left and right pelvic bones placed thereon; and a central shaft connected with the first correction part and the second correction part so that the first correction part and the second correction part can rotate.

The first correction part and the second correction part may include each connection ring to prevent separation of the first correction part and the second correction part from the central shaft. The each connection ring may be rotatably coupled to the central shaft in an up/down direction. The connection rings may be coupled to the central shaft by the same length so that the first correction part and the second correction part can rotate on the central shaft in the same radius.

The first correction part and the second correction part may include slanted portions which slant upwardly from the central shaft. The user's left and right pelvic bones may be placed on the first correction part and the second correction part.

The central shaft may include a supporting part coupled to the central shaft to prevent separation of the first correction part and the second correction part.

The pelvic balancer may further include a correction board formed with a center line to check whether a center of the user's left and right pelvic bones lies thereon. The central shaft may be provided at the middle of the center line. The center line may include a first center line extending along a horizontal axis and a second center line extending along a vertical axis so that the correction board can be used in both horizontal and vertical directions. The central shaft may be provided at an intersection of the first center line and the second center line.

### BRIEF DESCRIPTION OF THE DRAWINGS

FIG. 1 is an exploded perspective view of a pelvic balancer according to an embodiment of the present invention;

FIG. 2 is a perspective view of the pelvic balancer according to an embodiment of the present invention;

FIG. 3 is a plan view of the pelvic balancer according to an embodiment of the present invention;

FIG. 4 is a side view showing a state of using the pelvic balancer according to an embodiment of the present invention; and

FIG. 5 is a plan view showing a state of using the pelvic balancer according to an embodiment of the present invention.

### DETAILED DESCRIPTION OF THE EMBODIMENTS

Embodiments of the present invention will be described in detail with reference to the annexed drawings.

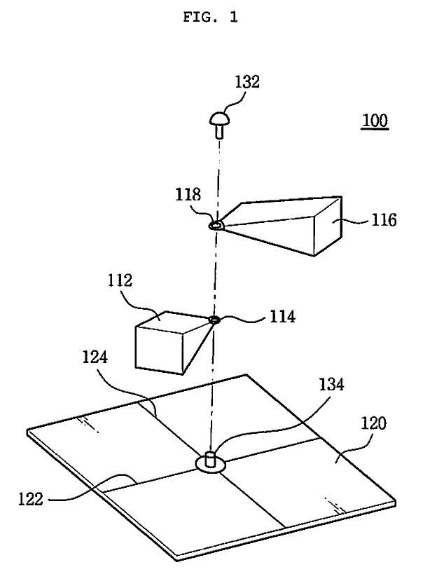

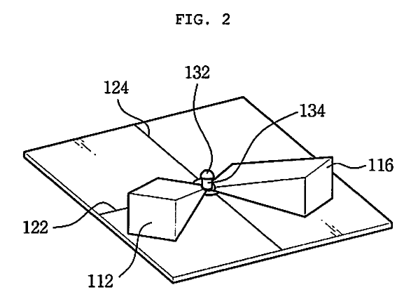

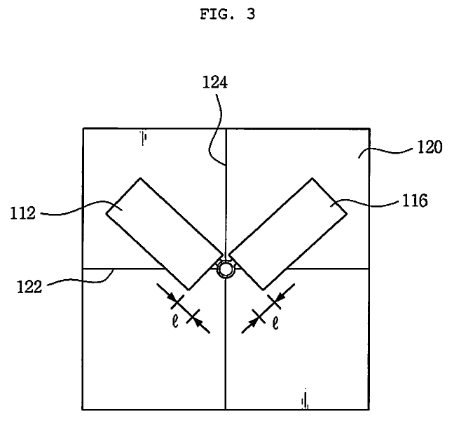

Referring to FIGS. 1 to 3, a pelvic balancer 100 according to an embodiment of the present invention includes a first correction part 112, a second correction part 116, and a central shaft 134. The first correction part 112 and second correction part 116 are used on the left and right pelvic bone in either order. The central shaft is used to prevent separation of the first correction part 112 and the second correction part 116 and to maintain the same radius for these two parts. The pelvic balancer 100 may further include a correction board 120 on which a part of a user's body is placed.

The first correction part 112 and the second correction part 116, on which the user's left and right pelvic bones are placed, are provided in pairs and have substantially the same shape, each being formed in a trigonal prism shape (e.g., pie shaped or wedge shaped). The trigonal prism shaped first and second correction parts 112 and 116 are placed on one of its flat surfaces such that triangular portions become side surfaces of the trigonal prism shape. The correction parts 112, 116 are oriented such that a lowest portion of the trigonal prism shape is located proximate the central shaft 134, and a highest portion of the trigonal prism shape is located at a distance from the central shaft 134.

Therefore, each of the first correction part 112 and the second correction part 116 has a sloped surface which slants upwardly from the central shaft 134. When the user's left and right pelvic bones are placed on the first and second correction parts 112 and 116, the user's weight is applied to the pelvis. In order to increase a pelvis correction effect, the triangular portion of the trigonal prism shape may be configured as a right triangle or substantially thereto.

The maximum height of each of the first and second correction parts 112 and 116 may be set to 5 to 15 cm, preferably, 7 to 13 cm so as to support the pelvis in a balanced state while user's upper and lower parts of the body are in a stable state.

In order to avoid deformation of the correction parts 112, 116 by a user's weight, the first correction part 112 and the second correction part 116 are made of hard material, e.g., wood or plastic, that keeps its shape. The surfaces of the first and second correction parts 112 and 116, on which a user's body is placed, may be covered with a soft material, such as polyurethane, sponge, cotton, etc., for added comfort.

The surfaces of the first correction part 112 and the second correction part 116 may be covered with a material such as real leather or artificial leather, in order to remove a foreign body sensation or unpleasant sensation and to prevent slip.

The first correction part 112 and the second correction part 116 are coupled to the central shaft 134 in order to prevent separation. The first correction part 112 and the second correction part 116 have a first connection ring 114 and a second connection ring 118, respectively, so as to be rotatably coupled to the central shaft 134. The first connection ring 114 and the second connection ring 118 are rotatably connected to the central shaft 134 so that the first correction part 112 and the second correction part 116 can rotate on the central shaft 134. Also, in order to prevent separation from the central shaft 134, the first connection ring 114 and the second connection ring 118 are sequentially and fixedly coupled to the central shaft 134 in an up/down direction of the central shaft 134. Further, the first connection ring 114 and the second connection ring 118 are connected to the first correction part 112 and the second correction part 116, respectively, by the same length l so that the first correction part 112 and the second correction part 116 can rotate in the same radius.

Although not illustrated in the drawings, the first correction part 112 and the second correction part 116 may further include knobs (e.g., handles) to facilitate movement of the first correction part 112 and the second correction part 116. The knobs may be provided at upper portions of the first correction part 112 and the second correction part 116.

When a user's pelvis is placed on the first and second correction parts 112 and 116, the central shaft 134 prevents separation of the first correction part 112 and the second correction part 116 due to a user's weight. The central shaft 134 is connected with the first correction part 112 and the second correction part 116 with the same radius, in order to accurately check the position on which the pelvis is placed. The central shaft 134 is protrudingly formed so that the first connection ring 114 and the second connection ring 118 are rotatably connected (around the axis of the central shaft) and fixedly coupled to the central shaft 134 in the up/down direction.

The central shaft 134 may further include a supporting part 132 to prevent separation of the first connection ring 114 and the second connection ring 118. The central shaft 134 may include a screw thread, and the supporting part 132 may be configured as a nut. By the engagement between the central shaft 134 and the supporting part 132, the separation of the first connection ring 114 and the second connection ring 118 from the central shaft 134 is prevented.

The correction board 120 is formed with a center line to check whether the center of a user's body is aligned with the board. The central shaft 134 is placed in the middle of the center line. The center line may include a first center line 122, which extends along a horizontal axis, and a second center line 124, which extends along a vertical axis, so that the correction board 120 can be used in both horizontal and vertical directions. In such a case, the central shaft 134 is provided at the intersection of the first center line 122 and the second center line 124. Although it is illustrated in FIGS. 1 to 3 that the correction board 120 is formed in a rectangular shape, the shape of the correction board 120 is not limited to a rectangle. The shape of the correction board 120 has no limitation, so far as the correction board 120 is formed with the center line, the central shaft 134 is provided at the middle of the center line, and the first correction part 112 and the second correction part 116 can rotate on the central shaft 134 in a predetermined radius.

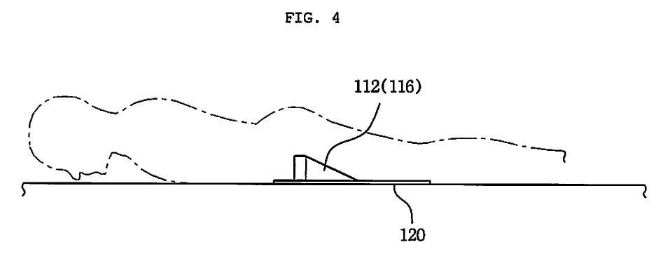

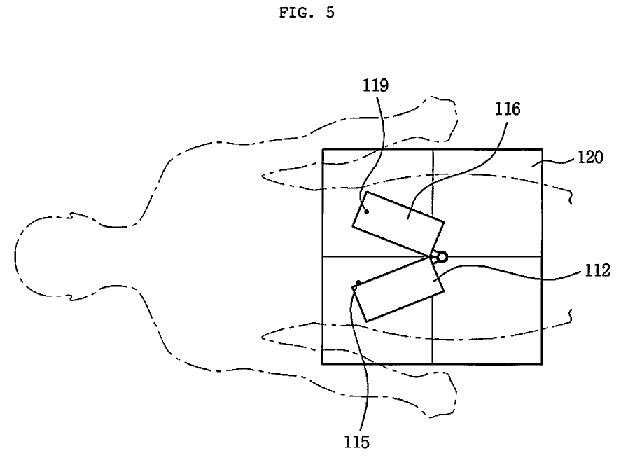

Hereinafter, a method of using the pelvic balancer according to the present invention will be explained in detail with reference to FIGS. 4 and 5.

Referring to FIGS. 4 and 5, first, a user lies prone with legs straight. Then, lengths of user's left and right legs are measured (e.g., from the hip bone to the heel of the foot) to check which leg is relatively long or short. Additional useful information may be obtained through clinical evaluation of the temporomandibular joints and neurological systems. Healthcare professionals may take additional measurements and fine-tune therapeutic positions of this device and user to enhance therapeutic effects.

Thereafter, a user lies prone on the pelvic balancer so that a user's center of body is positioned on the center line with her/his spinal column straight and a user's pelvis is positioned on the first correction part 112 and the second correction part 116. The first correction parts 112 is located anterior superior iliac spine of the relatively long leg and the second correction parts 116 is located anterior superior iliac spine of the relatively short leg. Then, the first correction part 112 and the second correction part 116 are first adjusted to be substantially at the same angle from the center line of the correction board 120.

Subsequently, the first correction part 112 and the second correction part 116 are secondarily adjusted so that user's hips are balanced and the left leg and the right leg are the same in length. In such a state, the user maintains a posture of lying prone on the pelvic balancer for a predetermined time.

If there is no length difference between the user's left and right legs, the anterior superior iliac spines of the left and right pelvic bones placed on the first and second correction parts 112 and 116 are positioned at a center point 119 on each correction part, which indicates that the left and right pelvic bones are balanced. However, if there is a length difference between the user's left and right legs, the anterior superior iliac spine of the pelvic bone of the relatively short leg placed on the correction part is located at a point 115 inward from the position where the anterior superior iliac spine of the pelvic bone of the relatively long leg placed on the other correction part.

Therefore, the user maintains a posture of lying prone on the pelvic balancer until the anterior superior iliac spine of the pelvic bone of the relatively short leg is positioned at the center point 119 on the correction part, similar to the anterior superior iliac spine of the pelvic bone of the relatively long leg. If the length difference between the user's short leg and long leg is large, the above-described medical treatment is repeatedly performed at regular intervals.

As described above, the pelvic balancer according to the present invention is structured such that the first correction part 112 and the second correction part 116 are formed to have the same shape and can rotate on the central shaft 134 in the same radius. Also, the center line is formed on the correction board, to easily check the balance of a user's body. Therefore, the angles of the correction parts can be easily adjusted, and the positions of the correction parts can be accurately checked. Accordingly, even an unskilled user can easily use the pelvic balancer according to the present invention. Further, since the correction parts are rotatably coupled to the central shaft and fixedly arranged in an up/down direction, the positions of the correction parts can be easily changed, and the correction parts are prevented from being separated from normal positions due to a user's weight or motion.

As apparent from the above description, the pelvic balancer according to the present invention has an effect of correcting misalignment of a pelvis in a short time while minimizing a user's pain by setting a dislocated pelvis to a normal position by using a user's weight, a lever system of the pelvic balancer supporting the misaligned pelvis placed thereon and a breathing mechanism without application of an external physical pressing force.

Further, since a left pelvic bone and a right pelvic bone are placed on the rotatable first correction part and second correction part, the left pelvic bone and the right pelvic bone are pressed by a user's weight, thereby correcting imbalance of the left pelvic bone and the right pelvic bone.

Further, since the first correction part and the second correction part are formed to have the same rotational radius and the center line is formed on the central shaft provided at the joining portion of the first correction part and the second correction part, to easily check whether the center of a user's body lies thereon, the left pelvic bone and the right pelvic bone can be set to normal positions only by simple manipulation without a sophisticated technique, thereby maximizing the pelvis correction effect.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pelvic balancer comprising:
a first correction part to support any one of user's left and right pelvic bones placed thereon;
a second correction part to support the other one of the user's left and right pelvic bones placed thereon; and
a central shaft connected with the first correction part and the second correction part, wherein the first correction part and the second correction part are rotatable around the central shaft.

2. The pelvic balancer according to claim 1, wherein the first correction part and the second correction part include each connection ring to prevent separation of the first correction part and the second correction part from the central shaft, the each connection ring being rotatably coupled to the central shaft.

3. The pelvic balancer according to claim 1, wherein the first correction part and the second correction part include each connection ring rotatably coupled to the central shaft,
and wherein each connection ring is coupled to the central shaft by the same length to rotate the first correction part and the second correction part in the same radius.

4. The pelvic balancer according to claim 1, wherein the first correction part and the second correction part include a sloped surface which slants upwardly from the central shaft, and
the user's left and right pelvic bones are placed on the first correction part and the second correction part.

5. The pelvic balancer according to claim 1, wherein the central shaft includes a supporting part coupled to the central shaft to prevent separation of the first correction part and the second correction part.

6. The pelvic balancer according to claim 1, further comprising a correction board formed with a center line to check whether a center of the user's left and right pelvic bones lies thereon,
and wherein the central shaft is provided at the middle of the center line.

7. The pelvic balancer according to claim 6, wherein the center line includes a first center line extending along a horizontal axis and a second center line extending along a vertical axis,
and wherein the central shaft is provided at an intersection of the first center line and the second center line.
